(19) United States
(12) Patent Application Publication
(10) Pub. No.: US 2003/0109756 A1
(43) Pub. Date: Jun. 12, 2003

(54) Process for the preparation of 3,3,5-trimethylcyclohexanol

(76) Inventors: Walter Kuhn, Holzminden (DE); Hans-Ulrich Funk, Lauenforde (DE); Gerhard Senft, Holzminden (DE); Heinz-Dieter Quest, Hoxter (DE)

Correspondence Address: BAYER POLYMERS LLC, 100 BAYER ROAD, PITTSBURGH, PA, 15205, US

(21) Appl. No.: 10/308,566

(22) Filed: Dec. 3, 2002

(30) Foreign Application Priority Data
Dec. 6, 2001 (DE) 10160009.7

(51) Int. Cl. C07C 35/08

(52) U.S. Cl. 568/822

(57) ABSTRACT

The invention relates to a process for the preparation of 3,3,5-trimethylcyclohexanol by hydrogenation of isophorone in the presence of a ruthenium catalyst.

### FIELD OF THE INVENTION

The invention relates to a process for the preparation of 3,3,5-trimethylcyclohexanol by hydrogenation of isophorone (3,3,5-trimethyl-2-cyclohexen-1-one) in the presence of a ruthenium catalyst.

### BACKGROUND OF THE INVENTION

3,3,5-Trimethylcyclohexanol is a flavoring with menthol-like character. (S. Arctander, Perfume and Flavor Chemicals, No. 2998, 1969 Montclair, N.J. USA).

3,3,5-Trimethylcyclohexanol can also used as starting material for the preparation of homomenthyl salicylate (3,3,5-trimethylcyclohexyl salicylate), which is used as a UV filter.

In J. Amer. Pharm. Assoc. 1942, 25, isophorone is hydrogenated in the presence of platinum at room temperature to give 3,3,5-trimethylcyclohexanol. The catalyst is used in the ratio 1:17.5 relative to isophorone.

In Chem. and Ind. 1933, 518 the hydrogenation of isophorone under Raney nickel catalysis or copper chromite catalysis is mentioned. A mixture of 3,3,5-trimethylcyclohexanol and 3,3,5-trimethylcyclohexanone is formed.

Neftepererab. Neftekhim. (Moscow) 1971, 5, 41 describes the hydrogenation of isophorone to give 3,3,5-trimethylcyclohexanol under Raney nickel catalysis at 15 to 140° C. The hydrogenation is carried out at a pressure of 35-100 bar and gives 3,3,5-trimethylcyclohexanol with 90% yield.

According to the described processes, 3,3,5-trimethylcyclohexanol can only be prepared with an unsatisfactory yield and/or using relatively large amounts of catalysts, some of which are also carcinogenic.

The object was therefore to find a process for the preparation of 3,3,5-trimethylcyclohexanol which can produce 3,3,5-trimethylcyclohexanol in a good yield and in high purity.

### SUMMARY OF THE INVENTION

Therefore, the present invention provides a process for the preparation of 3,3,5-trimethylcyclohexanol by hydrogenation of isophorone, characterized in that the hydrogenation is carried out in the presence of a catalyst comprising ruthenium.

### DETAILED DESCRIPTION OF THE INVENTION

The process according to the present invention permits, in particular, the preparation of sensorily perfect 3,3,5-trimethylcyclohexanol from an economic point of view, even on an industrial scale.

The 3,3,5-trimethylcyclohexanol prepared according to the invention can, for example, be used as flavoring and for the preparation of homomenthyl salicylate, a common UV filter.

The process according to the invention can be represented by the following reaction equation:

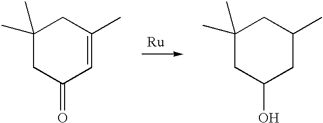

For the process according to the present invention, the catalyst can be used in the dry or moist state (water content up to 60% by weight).

The catalysts according to the present invention contains ruthenium in elemental, metallic form.

The ruthenium can, for example, be used in finely divided form, applied to supports or together with other metals (e.g. mixtures, alloys). The catalysts can contain dopings with one or more metals.

The ruthenium can be applied to organic or inorganic support materials. The catalysts can contain a support material or mixtures of support materials. Advantageous support materials which may be mentioned are: activated carbon, carbon, aluminum oxides, metal oxides, silica gels, zeolites, clays, clay granules, amorphous aluminum silicates or other inorganic supports. Preferred support materials are activated carbon, silicon dioxide, calcium carbonate and aluminum oxide. A particularly preferred support material is activated carbon.

A preferred catalyst is ruthenium on activated carbon.

The catalysts can be used as moldings, such as, for example, hollow strands, tablets, extrudates, spheres, cylinders, tubes, cones and the like, which is particularly advantageous if the reaction is carried out continuously.

If catalysts containing support materials are used, the proportion of ruthenium on the support material can generally be 0.5 to 50% by weight, preferably 1 to 20% by weight, and more preferably 3 to 10% by weight, based on the dry catalyst.

It is likewise possible to produce the ruthenium metal prior to the start or during the hydrogenation in situ by reduction with hydrogen from corresponding compounds, such as oxides or salts, in which case the ruthenium can optionally be precipitated onto a support. Suitable for this purpose are, for example, ruthenium tetroxide, ruthenium tetroxide hydrate and also ruthenium halides.

For the process according to the present invention, the weight ratio of isophorone to ruthenium metal is in the range 500,000 to 100:1, advantageously in the range 200,000 to 200:1, preferably in the range 50,000 to 500:1, more preferably in the range 30,000 to 5000:1.

The amount of ruthenium metal here refers to the absolute content of ruthenium, i.e. without support material and without any water or other constituents of the catalyst which may be present.

The process is carried out according to the invention at 30 to 220° C., preferably at 60 to 190° C. and more preferably at 90 to 160° C.

The process according to the invention is carried out with hydrogen, the hydrogen pressures are usually in the range 1 to 100 bar abs., and preference is given to carrying out the reaction at hydrogen pressures in the range 5 to 50 bar abs., in particular in the range 10 to 20 bar abs.

The hydrogenation time is usually in the range 2 to 100 hours, preferably in the range 5 to 40 hours.

The process can be carried out continuously, semicontinuously and batchwise.

The process according to the present invention can be carried out using solvents or solvent mixtures. Suitable are, for example, alcohols, aqueous alcohols, ethers, esters, aromatic or saturated hydrocarbons. For example, solvents such as methanol, ethanol, isopropanol, n-propanol, isobutanol, n-butanol, sec-butanol, tetrahydrofuran, dibutyl ether, ethylene glycol dimethyl ether, ethyl acetate, methyl acetate, pentane, hexane, heptane, octane, cyclopentane, cyclohexane, methylcyclohexane, cyclooctane, benzene, toluene, ethyl benzene or xylenes can be used.

The process is preferably carried out without a solvent.

The process according to the present invention can be carried out, for example, as follows.

Isophorone and the catalyst are initially introduced into a pressure vessel. Hydrogenation is carried out at the chosen temperature and the chosen hydrogen pressure. When the hydrogenation is complete, the crude 3,3,5-trimethylcyclohexanol can be obtained by removing the catalyst (e.g. filtration, decantation, centrifugation). If required, further purification of the 3,3,5-trimethylcyclohexanol can be carried out, for example by distillation.

By using the process according to the present invention, it is possible, depending the reaction conditions, to achieve various ratios of cis and trans isomers in the hydrogenation product. The ratio of cis:trans isomer is typically in the range from about 60:40 to 90:10. Particularly in the case of prolonged hydrogenation times, 3,3,5-trimethylcyclohexanol which has a high proportion of cis isomer is formed. A 57:43 isomer mixture (cis:trans) has a fresher taste compared, for example, with the known and also market-standard 90:10 isomer mixture.

The invention is further illustrated but is not intended to be limited by the following examples in which all parts and percentages are by weight unless otherwise specified.

### EXAMPLE

2,493 g of isophorone and 5.8 g of ruthenium on activated carbon (Ru content: 5% by weight, water content: about 55% by weight; the ratio of Ru to isophorone was accordingly approximately 1:19,000) were initially introduced into a 5 l stirred autoclave with gas-dispersion stirrer. Hydrogenation was carried out for 7.5 hours at 140° C. and a hydrogen pressure of 18 bar. After the hydrogenation, 2,553 g of 3,3,5-trimethylcyclohexanol with a GC purity of 99.7% by weight were obtained. The resulting 3,3,5-trimethylcyclohexanol can, if required, be distilled to the greatest possible extent without residue at a bottoms temperature of 130° C. and 10 mbar. The cis:trans isomer ratio was 92:8.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the preparation of 3,3,5-trimethylcyclohexanol comprising the step of hydrogenating isophorone, wherein hydrogenation is carried out in the presence of a catalyst comprising ruthenium.

2. A process according to claim 1, wherein said catalyst is ruthenium on activated carbon.

3. A process according to claim 1, wherein the weight ratio of isophorone to ruthenium is in the range 500,000 to 100:1.

4. A process according to claim 1, wherein hydrogenation is carried out at a temperature in the range from 30 to 220° C.

5. A process according to claim 1, wherein hydrogenation is carried out at a hydrogen pressure of from 1 to 100 bar abs.

6. A process according to claim 1, wherein hydrogenation is carried out in the absence of a solvent.
